(19) United States
(12) Patent Application Publication
(10) Pub. No.: US 2015/0065812 A1
(43) Pub. Date: Mar. 5, 2015

(54) TELEMEDICINE INFORMATION SYSTEM, MONITORING METHOD AND COMPUTER-ACCESSIBLE STORAGE MEDIUM

(71) Applicant: EBM TECHNOLOGIES INCORPORTED, Taipei City (TW)

(72) Inventor: WILLIAM PAN, TAIPEI CITY (TW)

(73) Assignee: EBM TECHNOLOGIES INCORPORTED, Taipei City (TW)

(21) Appl. No.: 14/016,213

(22) Filed: Sep. 2, 2013

(51) Int. Cl. A61B 5/00

(52) U.S. Cl. CPC A61B 5/0022
USPC 600/300

(57) ABSTRACT

Disclosure herein is related to a telemedicine information system, a monitoring method, and computer-accessible storage medium. The telemedicine information system includes at least one client end in which one or more physiological sensors for sensing the physiological conditions of a person to be monitored. The system includes a monitoring end connected to the client end over a network for receiving physiological data therefrom. Medical staff at the monitoring end uses a handheld monitoring terminal device to retrieve the physiological data. Then a program is executed to determine any abnormal condition. The medical staff may therefore issue a warning signal to the client end, and also timely communicate with the person to be monitored. The person may also timely receive location-based medical advice.

### BACKGROUND OF THE INVENTION

1. Field of the Invention

The present invention is generally related to a medical system, a monitoring method, and a computer-accessible storage device; more particularly to a telemedicine information system which disposes a mobile device at a monitoring end for anytime obtaining physiological condition of the client end, and timely issuing a warning signal.

2. Description of Related Art

With the enhancement of network bandwidth and well development of network technology, the various related applications are widespread to the general users. For example, in order to provide health care to the users, the network technology may be applied to use of telemedicine at a distance. That means the telemedicine provides the users at home the health care at a distance, including the any medical aid from the remote medical staff.

The kind of telemedicine technology may incorporate the modern computer network technologies. The telemedicine may be associated with the computer, communication, and audio/video processing technologies. The telemedicine provides the medical care at a distance, and broader range of medical service made by the medical institutions since it combines the well-developed network technology and eh medical profession. In particular, the service is directed to the disabled persons, or the persons at regions that are inaccessible and lack of medical facilities.

The general telemedicine service is such as voice/video call for remote interrogation, remote care, television monitoring, or even the doctor's diagnosis made according to physiological data retrieved from the client end's monitoring equipment.

### SUMMARY OF THE INVENTION

Disclosure in accordance with the present invention is related to a telemedicine information system which provides the medical staff to utilize a handheld device so as to monitor physiological condition at one or more client ends. A physiological monitor at the client end is used to measure and generate physiological chart and message regarding the client. Therefore, the medical staff may remotely acknowledge the client's physiological condition. When any emergency happens, the monitor may timely issue warning signal for help.

To observe the beneficial result to the person to be monitored, the present method provides the effective solutions to the long-felt need for the remote and instant help to the patients. For example, when the person may not easily ask for physiological monitoring since he is at vacation in a foreign country, the present method is one of the solutions. Other conditions such as the patients who require long-term health rehabilitation or medical care as any emergency occurs at home care.

The disclosure is related to a telemedicine information system, a monitoring method, and a computer-accessible storage device. The invention is based on the present network technology and provided for performing a remote medical care.

In an exemplary embodiment of the telemedicine information system, the system includes at least one client end, which is equipped with one or more physiological sensors that are used to monitor physiological conditions of the person to be monitored. The sensors generate physiological data for a monitoring end to perform monitoring. The monitoring end is connected to the one or more clients over a network. Through a monitoring server, the monitoring end receives the physiological data from the client ends.

At the monitoring end, the medical staff manipulates a monitoring terminal device. The monitoring terminal device is such as a handheld communication set. Over the network, the monitoring server may receive the physiological data. A monitoring program is executed in the monitoring terminal device, and provided to automatically determine if there is any abnormal condition according to a judgment rule.

In one aspect, the monitoring end may have a subscriber database used to record the physiological data transmitted from the client end. The data may then be accessed by the monitoring terminal device. It is noted that the monitoring terminal device and the monitoring server are within a wireless local network, and can also be at a remote network. The network-based technology allows the monitoring terminal device to access the physiological data of the subscriber database through the monitoring server.

In one further aspect, a communication set at the client end is allowed to communicate with the medical staff at the monitoring end, and receive messages.

The monitoring method in one embodiment includes firstly a program being executed in the monitoring terminal device. While entering the application, a user privilege may be firstly verified by this program. Next, the program is to drive the monitoring terminal device to establish a connection with a monitoring server. The program may then be allowed to access a subscriber database. According to the user privilege, the program may access information from the client end. In particular, the monitoring terminal device may display the information of client end on a screen. This program may automatically judge if there is any abnormal condition.

Once the program recognizes an abnormal condition from the physiological data, a communication method may be driven to send message to the client end, or alternatively make a voice or video call for the client end with the medical staff at the monitoring end.

The invention is also directed to a computer-accessible storage device. The storage device stores an instruction set for instructing the mentioned telemedicine monitoring method. The instruction set includes instruction for affirming user privilege for operating the telemedicine monitoring; instruction for establishing connection to a monitoring server; instruction for accessing information of client end according to user privilege; instruction for displaying information onto a display; and instruction for determining if any abnormal condition from the physiological data.

### BRIEF DESCRIPTION OF THE DRAWINGS

The foregoing aspects and many of the attendant advantages of this invention will be more readily appreciated as the same becomes better understood by reference to the following detailed description, when taken in conjunction with the accompanying drawings, wherein:

FIG. 1 schematically shows a client and a monitoring end in the telemedicine information system according to one embodiment of the present invention;

FIG. 2 shows a schematic diagram illustrating facilities at a monitoring end of the telemedicine information system according to one embodiment of the present invention;

FIG. 3 shows a schematic diagram illustrating the telemedicine information system including a client end and a monitoring end according to one embodiment of the present invention;

FIG. 4 shows a flow chart illustrating a monitoring method in the telemedicine information system according to one embodiment of the present invention;

FIG. 5 schematically shows facilities at a client end and a monitoring end of the telemedicine information system according to one embodiment of the present invention;

FIG. 6 shows a schematic diagram depicting an information interface of a monitoring terminal device;

FIG. 7 schematically shows a chart depicting monitoring information at a monitoring terminal device;

FIG. 8 schematically shows an interface depicting user information at a monitoring terminal device.

### DETAILED DESCRIPTION OF THE PREFERRED EMBODIMENTS

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

At least one of the objectives of the telemedicine information system of the present invention is to provide an efficient, instant, and convenient telemedicine environment. The telemedicine information system renders a monitoring method that allows medical staff to use a handheld device to monitor patient's physiological chart and messages. Especially, the physiological data is made by a physiological monitor at a client end. Through this monitoring method, the medical staff may remotely monitor the patient's condition at the client end instantly from the monitoring end. Therefore, the system timely facilitates medical assistance while any emergency occurs with instant warning signal.

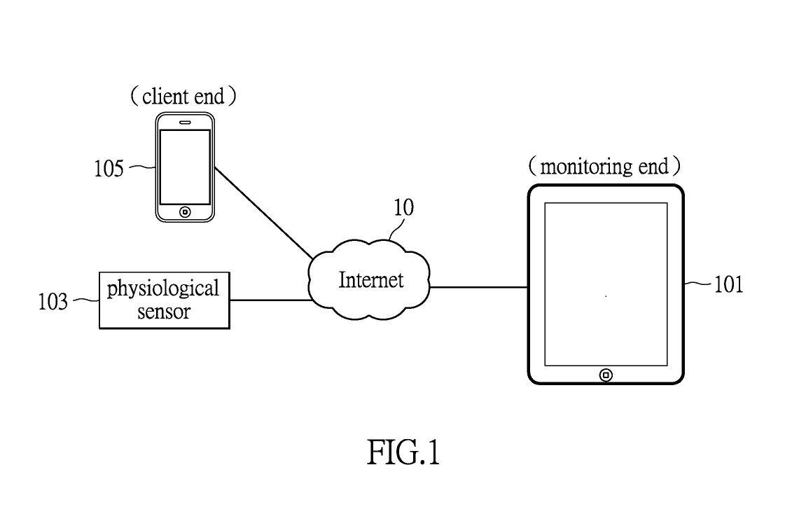

The exemplary framework of the telemedicine information system in accordance with the present invention is referred to FIG. 1. In this schematic diagram, a relationship between a client and a monitoring end of the telemedicine information system is illustrated.

The monitoring end is connected with at least one client end over a network such as Internet 10. The monitoring end may anytime recognize physiological data retrieved from multiple client ends.

In an exemplary example, the medical staff at the monitoring end is such as doctor or/and nurse who is equipped with at least one monitoring terminal device 101. Preferably, the monitoring terminal device 101 is such as a handheld tablet, smart phone, or any computer device that has a display and capability of communication. The physiological data at the client end may be retrieved by a monitoring program operated in the monitoring terminal device 101. The program is able to make diagnosis of the physiological and judge if any abnormal condition according to a judgment rule.

In the figure, a physiological sensor 103 appeared to sense physiological condition for the person to be monitored is installed at the client end. For example, the physiological sensor 103 is such as a heart rhythm sensor, a sphygmometer, oximeter, respiratory frequency measurement instrument, or thermometer. One or in combination of more than one sensor may be installed as required. In general, these instruments have no capability of communication, or with limited functions. However, the telemedicine information system requires the physiological sensors able to link to network, or conduct the communication with plug-in functionality. Alternatively, a communication set 105 at the client end may be configured to make the communication.

If the monitoring end is located remotely according one of the embodiments, the client end may connect with the monitoring end over Internet 10, so as to receive the various signals of physiological sensors. The staff at the monitoring end may monitor the person at the client end through the monitoring terminal device 101. Further, the device 101 allows the staff at the monitoring end to communicate with the person at the client end.

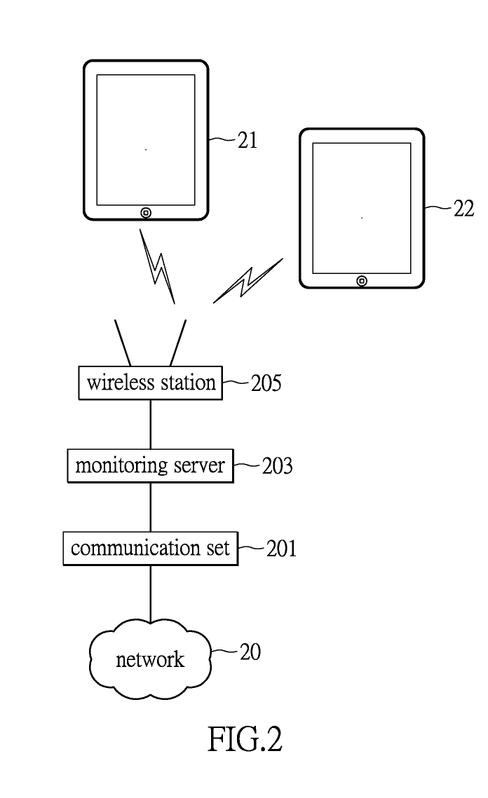

Reference is made to FIG. 2, which depicts facilities at the monitoring end of the telemedicine information system in accordance with the present invention.

A monitoring server 203 is implemented by a computer system. This server 203 includes a database used to record the physiological data retrieved from the client end over a network 20. The database records the various physiological data corresponding to the different physiological sensors/facilities, the patient's data, and his pathological data. The pathological data is such as the medical history, doctor's diagnosis record of the person or the patient. At the monitoring end, over the network 20, a communication set 201 serves as the device to bridge the network 20. Medical staff at the monitoring end or the operators may hold the monitoring terminal devices 21, 22 to connect with the monitoring server 203 over a local wireless station 205. Via the monitoring server 203, the staff may be permitted to access the client information and the corresponding physiological data from the database. Furthermore, via the monitoring server 203, the instantaneous physiological data from the client end may also be retrieved.

In an exemplary embodiment, the shown monitoring terminal devices 21, 22 are such as handheld computer device in which programs are installed. The programs preferably include a remote monitoring program adapted to the present invention. The communication circuits are driven to establish connection to the monitoring server 203 when the operating system of the monitoring terminal device 21 or 22 is operated to execute the monitoring program. Based on identification of the operator, or a privilege, the data of client end may be accessed. The client data may include ID and physiological data of each client. The monitoring program may provide function to automatically determine if there is any abnormal condition from the physiological data. The various indicative methods such as flashing, pictorial, vibration, and diagrammatic are used to notice the medical staff.

Still further, the monitoring terminal device 21, 22 allows the medical staff to communicate with the monitored person at the client end. The communication may be conducted by voice call or video call in order to render a location-based medical advice.

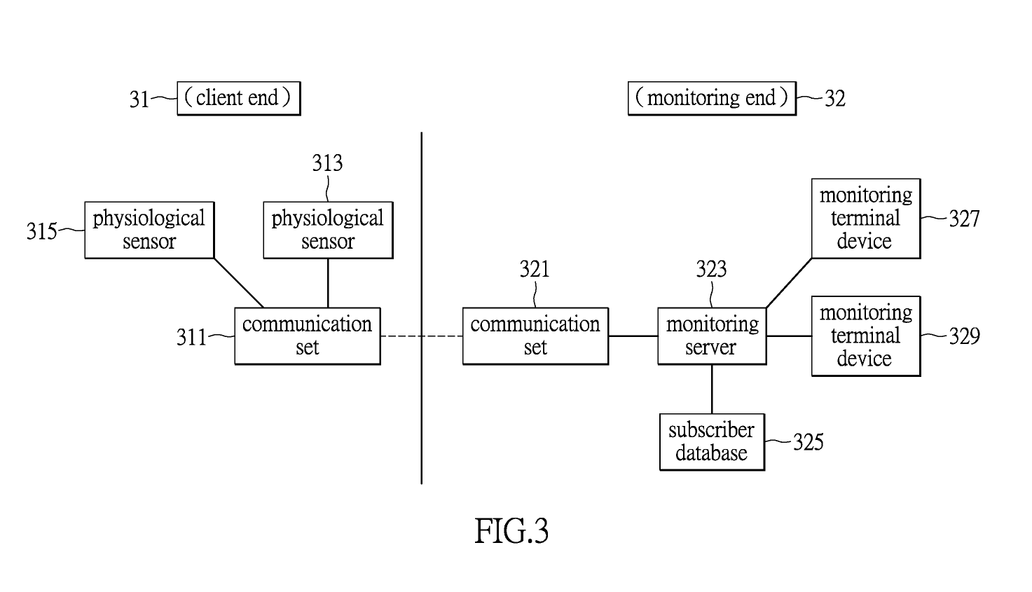

FIG. 3 depicts a connection relationship between the client and the monitoring end of the telemedicine information system according to one embodiment of the invention.

The system essentially includes a client end 31 and a monitoring end 32. At the client end 31, a communication set 311 conducting the communication and its connected physiological sensors 313, 315 are included. The communication set 311 may be a mobile communication device such as mobile phone besides the general network equipment such as the network router. The communication set 311 is used to transmit the signals made by the physiological sensors 313, 315, and the related message may be delivered or displayed onto the other monitoring terminal devices. Further, the signals associated with the physiological sensors 313, 315 may also be integrated for delivery. The example described in the figure may not substantially limit the invention. Alternatively, the physiological sensors 313, 315 may transmit the messages to the monitoring end 32 by their built-in communication modules.

At the monitoring end 32, the monitoring server 323 is configured to collect messages sent from the every client end 31. Further, this monitoring server may also be a platform serving communication means between the staff at the monitoring end 32 and the client end 31. The monitoring server is such as a platform for delivering any message made by video, audio or/and text. The shown monitoring server 323 is communicated with the facilities at the client end 31 via the communication set 321 at the monitoring end 32. More, an encrypted network channel may be established there-between in consideration of information security.

At the monitoring end 32, a subscriber database 325 is configured to record the data for the client end. The subscriber database 325 may include a variety of physiological data for the client end. The monitoring terminal devices 327, 329 are utilized to access the user information in the database 325 within the local network.

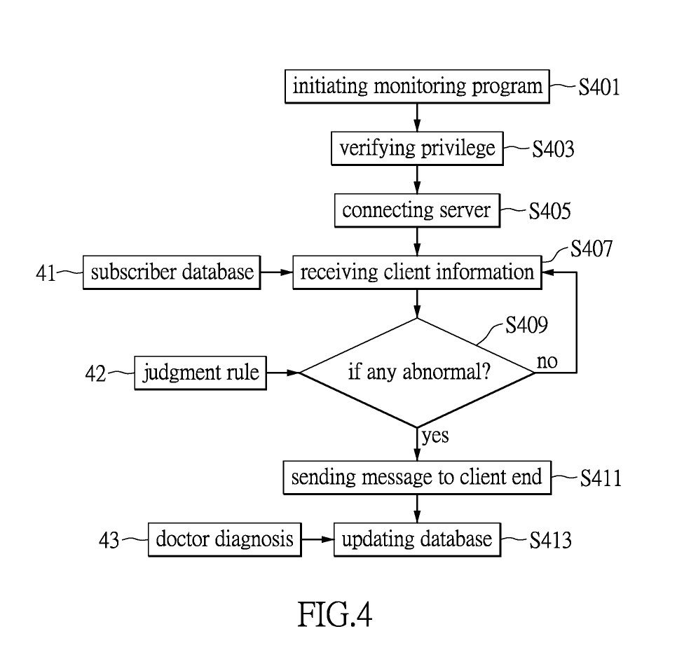

FIG. 4 next shows a flow chart describing a monitoring method for the telemedicine information system according to the embodiment of the invention. This flow may simultaneously be an operating process made by a monitoring program installed in the monitoring terminal device.

The monitoring end of the telemedicine information system is configured to install the monitoring terminal device provided for the medical staff or other operators to browse the physiological data. The monitoring terminal device is exemplarily a computer device capable of communicating and displaying. For example, a tablet computer or other portable device may be used to perform an instruction set, stored in memory, for embodying the monitoring method. The flow begins in step S401, a monitoring program is initiated. This program may firstly verify a user's identification. Because the monitoring terminal device is manipulated by some different users, the user of the monitoring program may be firstly identified before initiating to access the monitoring server. Furthermore, the authorized medical staff may be permitted to access the limited client data since he/she may serve some specific clients or patients.

The related privilege is then affirmed through any one of the various ways to identify the user in step S403, for example using an account and its corresponding password. The program therefore guides the user to select or automatically drive the circuit of the terminal device to establish a connection with a specified server (step S405). The connected server may be the mentioned monitoring server that may be within the same local network such as the embodiment described in FIG. 2, or at a remote external network.

The monitoring server includes a built-in subscriber database or links to an external database according the embodiment of the invention. In step S407, the monitoring terminal device may receive client data from the subscriber database 41 after the server is connected, preferably using the program in the device. The monitoring program is able to access the client data based on limited privilege while user identification is done. Such as step S407, the one or more client data may be accessed according to the user privilege. The retrieval of the client data may then be displayed onto a screen of the monitoring terminal device.

The data retrieved from the client end at least includes ID information of the client end, and the physiological data. The physiological data includes the monitoring data instantaneously retrieved from the client end, the physiological data within a period of time, or historical data. The data may be stored or to update the subscriber database 41. The authorized medical staff may modify the diagnosis content or annotate it before storing.

Next, such as step S409, the monitoring program determines if the message includes abnormal or normal conditions automatically when the monitoring terminal device retrieves physiological data form the every client end. A warning message may be remarked or issued when any abnormal condition is determined. The program may automatically determine if there is any abnormal condition. In the meantime, if no any abnormal condition is found, the step in the method goes to step S407 for continuously retrieving the physiological data and operate the judgment in addition to make record. On the contrary, if any abnormal condition is checked, the step goes to step S411 for the monitoring program issuing message to the client end, or for the medical staff to decide a way to communicate with the client. At last, the database is updated when the doctor makes a diagnosis (43) (step S413). It is noted that the steps of issuing the message presenting any abnormal condition and updating database never ends until all the physiological data are completely checked.

The subsequent operations may be such as the monitoring end notifying the client end with messages over a network as it meets any abnormal condition. However, the notification may also be the types of audio/video or/and texts. For example, the notification may advise the person to be monitored that he should go to the hospital.

In further embodiment, the monitoring end may acknowledge the location of the person to be monitored if the person holds a communication set with positioning function. For example, the monitoring program executed in the monitoring terminal device may acquire the positioning information set in the communication set at the client end. Based on this positioning information, the monitoring end may advise the person of any location-based medical advice such as recommendation for the neighboring medical institutions.

When the person to be monitored under the telemedicine information system is a patient who is bedridden in bed, he or she may wear the wearable physiological sensor for a period of time; the physiological sensor may periodically send the physiological data as the sensor is always on line. The monitoring end therefore periodically receives the message from the client end. Any medical decision can be made if any abnormal condition is checked.

In one further embodiment, the person to be monitored may wear the physiological sensor at the moment he/she feels unwell since the physiological sensor is not always put on. Therefore, the physiological data may be generated and sent to the monitoring end at the moment the person put on the sensor. The person to be monitored may activate a communication with the medical staff as he requires. The medical staff at the monitoring end may clearly check the medical data associated with the retrieved physiological data through his/her handheld monitoring terminal device. The staff may provide location-based medical advice to the person, or/and establish a communication channel to the client end. The advice is such as teaching any person at the client end to handle the emergency, or to take medicine. In an exemplary example, multiple medical staffs may simultaneously in consultation over a voice call or video call as a conference call. A suitable nearby hospital may be advised when the monitoring end can position the person to be monitored.

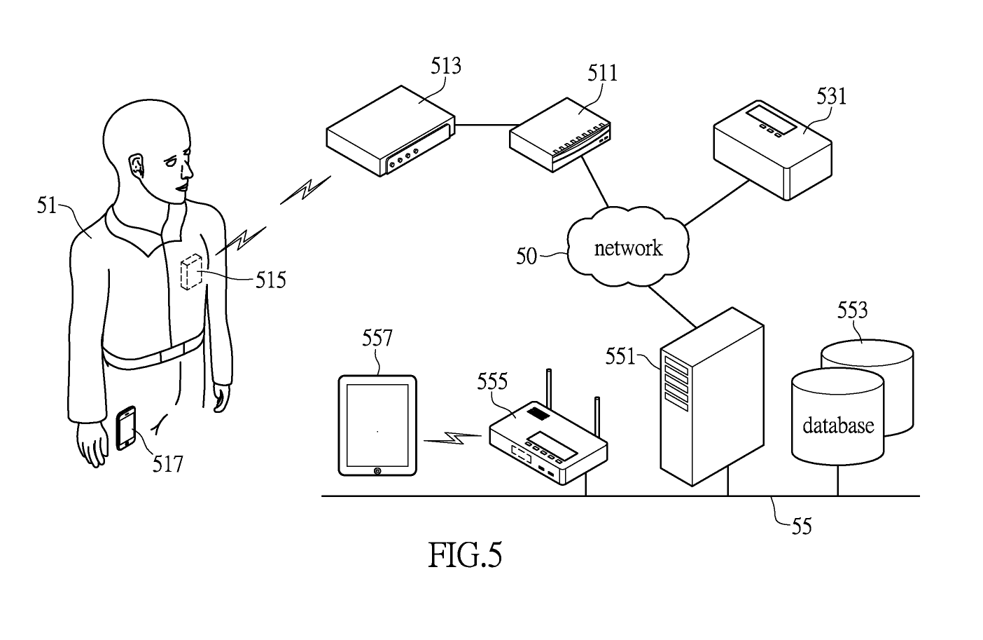

Reference is next made to FIG. 5 schematically depicting the relationship between the client end and the monitoring end in the telemedicine information system.

According to the figure, a to-be-monitored person 51 who wears a physiological sensor is at the client end. The physiological sensor is such as a heart rhythm sensor 515 which is used to measure the heart rhythm. This heart rhythm sensor 515 is connected with a signal receiver 513, which is used to collect the physiological signals from the various sensors. The facilities at the client end may get on network 50 via the network facility 511 for transmitting the physiological data.

In the current example, the person 51 to be monitored person holds a communication set 517, for example a mobile phone. If any abnormal condition is found by the monitoring end, the communication set 517 is utilized to communicate with the remote medical staff over a voice call, video call or by texts. In one further embodiment, the positioning circuit in the communication set 517 assists the monitoring end to recognize the person's location since he/she is permitted free to walk or go out. The positioning function allows the medical staff anytime to acknowledge the person's location. It is also convenient that the monitoring end sends staff to the site if any abnormal condition is checked. Any location-based medical advice may also be provided if the person needs.

In one embodiment, the communication set at the client end is such as a smart phone or tablet computer. The program at the monitoring end is allowed to send a navigation map to the communication set at the client end according to the positioning data. Therefore, the person at the client end may accordingly find the help in accordance with the navigation map.

At the monitoring end, the servers and devices are within a local network 55. The server at the monitoring end is such as a monitoring server 551 used for retrieving physiological data from the client end. A subscriber database 553 at the monitoring end is used to store and record information of the client end. A wireless station 555 is introduced to the system for rendering wireless communication service for the monitoring terminal device 557. The device 557 is therefore allowed to wirelessly access the monitoring server 551 and the subscriber database 553.

In this architecture of telemedicine information system, the client end may be accessible to the monitoring end over the network 50 in addition to the traditional telecommunication. Over the network 50, the both sides may be conducted with voice or audio/video call if any abnormal condition is found. The mentioned notification may be incorporated to this system of notifying message over the network 50. In an exemplary embodiment, a user interface may be initiated by the program operated in the monitoring terminal device 557, and used to display the message onto the screen of the device 557. The user interface may also guide the user, e.g. the medical staff, to operate the system, including network connection, retrieval of the physiological data, and the communication made to the client end. Besides the software program installed in the communication set, a program add-on in web browser or any specific web site may serve the foregoing functionalities.

In one embodiment of the system, a message server 531 is installed. This message server 531 renders the function of transferring messages. For example, the message server 531 may render the above-mentioned service of notification, by which the messages are transferred to the corresponding client end. While the message server 531 serves as an email server, the messages are the electronic mails. While the message server 531 conducts the short message service (SMS), the message server 531 may be a simple message service server.

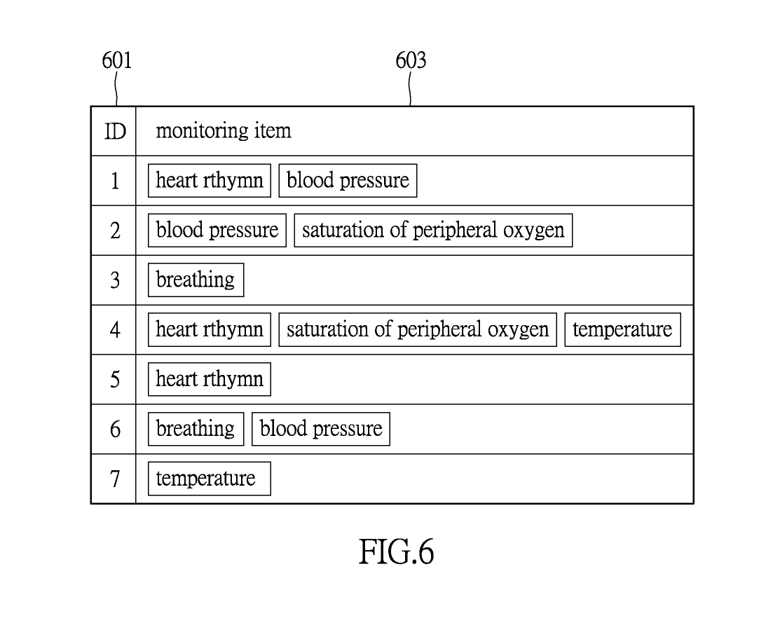

FIG. 6 schematically shows a user information screen displayed onto the monitoring terminal device.

This figure depicts the user interface of the monitoring terminal device. When the monitoring terminal device is utilized to retrieve client data associated with one or more client ends from a monitoring server, one or more monitored items 603 of the every client end may be displayed according its corresponding identification code 601. For example, the monitored items corresponding to the identification code 1 provided by the system are such as heart rhythm and blood pressure. The items corresponding to the identification code 2 are such as blood pressure and saturation of peripheral oxygen. The item for the client end with identification code 3 is such as breathing. Some other exemplary items may be clearly referred to the figure.

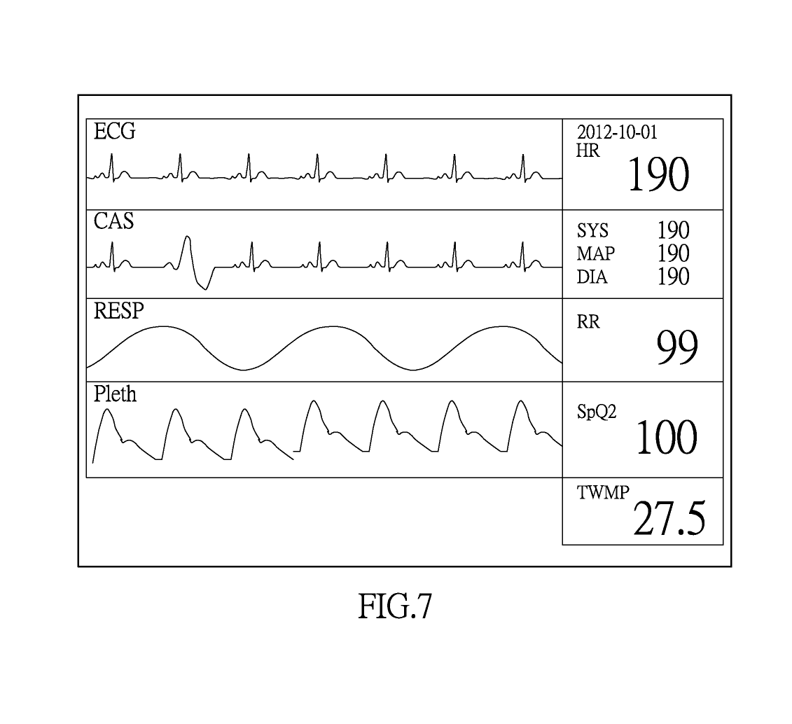

Reference is made to FIG. 7 which depicts a monitoring screen displayed on to the monitoring terminal device. One region of the user interface may be used to show the rich messages for one corresponding client end, for example an Electrocardiography (ECG) extracted from physiological data generated by an Electrocardiography machine. The result is then represented as the waves shown in the Electrocardiography. The monitoring program may provide function of alerting when any signal exceeds a specific threshold. The other applicable histogram is such as brain wave (CAS), respiratory (RESP), plethysmography (pleth) for judging cardiopulmonary condition, displaying SpO2, breathing frequency (RR), heart rate (HR), systolic blood pressure (SYS), diastolic blood pressure (DIA), and their average.

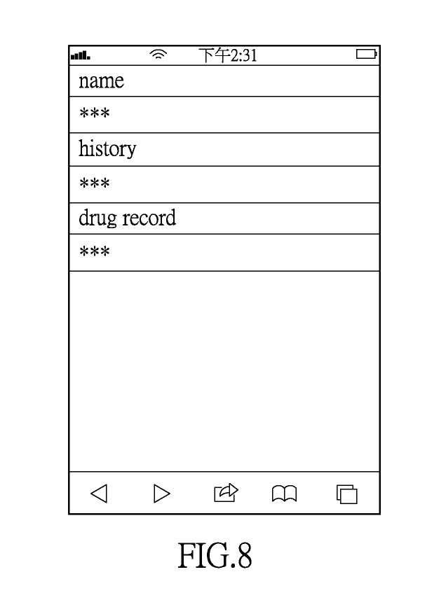

FIG. 8 further shows the client data displayed on to a computer display, and the information indicates the client's name, medical history and medication records.

To sum up the above description, the telemedicine information system in accordance with the present invention provides a terminal device held by the medical staff at a monitoring end to monitor physiological data retrieved from a client end. The terminal device may be installed with a program used to determine any condition extracted from the physiological data. Alerting message may be automatically issued if any abnormal condition is found. The medical staff may instantly make judgment and perform communication with the client for providing location-based medical advice.

The above-mentioned descriptions represent merely the preferred embodiment of the present invention, without any intention to limit the scope of the present invention thereto. Various equivalent changes, alternations or modifications based on the claims of present invention are all consequently viewed as being embraced by the scope of the present invention.

What is claimed is:

1. A telemedicine information system, comprising:
at least one client end, disposed with one or more physiological sensors at one client end, wherein the one or more physiological sensors are used to obtain physiological condition of a person who is to be monitored, and generate physiological data; and
a monitoring end, connected to the at least one client end over a network, via a monitoring server receiving the physiological data over the network, and via a monitoring terminal device obtaining the physiological data, so as to determine any abnormal condition according to a judgment rule.

2. The system of claim 1, wherein, when the client end receives physiological data, the data is firstly stored in a subscriber database, and accessible to the monitoring terminal device.

3. The system of claim 2, wherein the monitoring terminal device is connected to the monitoring server over a wireless LAN, and the physiological data is obtained from the subscriber database via the monitoring server.

4. The system of claim 3, wherein the monitoring terminal device is equipped with a memory, which is used to store programs executed for accessing the subscriber database.

5. The system of claim 1, wherein the client end is further equipped with a communication set, and the person to be monitored receives messages from the monitoring end through the communication set.

6. The system of claim 5, wherein the monitoring end transmits the messages to the client end by a network-based notification technology.

7. The system of claim 5, wherein the monitoring end obtains location of the person to be monitored through a positioning service made by the communication set, so as to provide location-based medical advice.

8. A telemedicine monitoring method, comprising:
a monitoring terminal device executing a program stored in a memory;
affirming a user privilege of the program;
by the program, driving the monitoring terminal device to connect to a monitoring server;
the monitoring server linking a subscriber database, by the program, retrieving one or more data of the client end via the monitoring server based on the user privilege and displaying the data on a display of the monitoring terminal device; wherein the one or more data include identification and physiological data of the client end; the physiological data is generated by one or more physiological sensors at the client end, and recorded in the subscriber database; and
displaying normal or abnormal condition based on the physiological data.

9. The method of claim 8, wherein, if the physiological data shows abnormal condition, a communication means is incorporated to transmitting related message to the client end.

10. The method of claim 9, wherein, by the communication means, medical staff at the monitoring end is allowed to perform a voice call or a video call with the client end.

11. The method of claim 10, wherein a judgment is recorded in the subscriber database after the medical staff makes the judgment.

12. The method of claim 9, wherein the communication means is network-based notification technology, and received by a communication set at the client end.

13. The method of claim 8, wherein both the monitoring terminal device and the monitoring server are within a LAN, and connected over a wireless connection.

14. The method of claim 8, wherein the program initiates a user interface displayed on the display of the monitoring terminal device.

15. The method of claim 14, wherein the user interface is used to display the data related to the client end and physiological data generated by the one or more physiological sensors at the client end.

16. The method of claim 8, further comprising, by the program, obtaining positioning information of a communication set held by the person to be monitored at the client end, and providing location-based medical advice.

17. The method of claim 16, wherein, by the program, a navigation map is transmitted to the communication set when the positioning data of the communication set is received.

18. A non-transitory computer-accessible storage device, used to store an instruction set which is used to execute a telemedicine monitoring method according to claim 8, wherein the instruction set comprises:
instruction for affirming user privilege of executing the instruction set performing the telemedicine monitoring;
instruction for connecting to a monitoring server;
instruction for obtaining data of one or more client ends based on the user privilege;
instruction for displaying the data on a display, wherein the data at the one or more client ends include identification and physiological data at the each client end; and
instruction for determining if there is any abnormal condition according to the physiological data.
